(19) United States
(12) Patent Application Publication
(10) Pub. No.: US 2007/0149946 A1
(43) Pub. Date: Jun. 28, 2007

(54) Advancer system for coaxial medical devices

(76) Inventors: Raju R. Viswanathan, St. Louis, MO (US); William Flickinger, Lino Lakes, MN (US)

Correspondence Address: Bryan K. Wheelock, Suite 400, 7700 Bonhomme, St. Louis, MO, 63105, US

(21) Appl. No.: 11/634,338

(22) Filed: Dec. 5, 2006

Related U.S. Application Data
(60) Provisional application No. 60/748,042, filed on Dec. 7, 2005.

(51) Int. Cl. A61M 5/178; A61M 31/00; A61M 25/00; A61B 5/00

(52) U.S. Cl. 604/500; 604/164.01; 600/585

(57) ABSTRACT

A telescoping medical device system is provided that includes an outer sheath having a proximal end and a distal end, and a lumen extending through at least the distal end portion, and a slot extending along a portion of the outer sheath. The telescoping medical device also includes an inner member extending through the slot and through the lumen in the outer sheath. A first drive mechanically engages and drives the outer sheath, and a second drive mechanically engages and drives the inner member through the slot and into the lumen of the outer sheath.

### CROSS-REFERENCE TO RELATED APPLICATIONS

This application claims the benefit of U.S. Provisional Patent Application Ser. No. 60/748,042, filed Dec. 7, 2005, the entire disclosure of which is incorporated herein by reference.

### BACKGROUND

The present disclosure relates to advancer systems for medical devices, and in particular to advancer systems for separately controlling the elements of a set of coaxially used medical devices.

The statements in this section merely provide background information related to the present disclosure and may not constitute prior art.

A variety of elongate medical devices may be navigated through the body to perform diagnostic and therapeutic procedures. The devices are most commonly manually advanced and retracted. Advancer systems have been developed for automatically advancing and retracting medical devices in the body. U.S. patent application Ser. No. 10/138,710, filed May 3, 2002, for System and Methods for Advancing a Catheter, or U.S. patent application Ser. No. 10/858,485, filed Jun. 1, 2004, for System and Methods for Medical Device Advancement, disclose examples of such devices. The problem of controlling coaxial medical devices in which two or more coaxial devices telescope relative to each other is more difficult. The geometry of such systems makes an advancer system for such devices more complex. U.S. Pat. No. 6,726,675 is an example of an attempt to resolve some of these difficulties.

Automation of device movement allows the physicians to perform the procedures remotely, which reduces exposure to radiation from x-ray imaging, and also facilitates telemedicine in which specialists in remote locations can conduct or at least supervise local procedures. These and other features and advantages will be in part apparent, and in part pointed out hereinafter.

### SUMMARY

The exemplary embodiments of the present invention facilitate movement of telescoping medical devices within a subject body. Some embodiments for facilitating movement of medical devices enable advancement or retraction of an outer member and an inner member either separately or together. In one embodiment, a telescoping medical device system is provided that includes an outer sheath having a proximal end and a distal end, and a lumen extending through at least the distal end portion, and a slot extending along a portion of the outer sheath. The telescoping medical device also includes an inner member extending through the slot and through the lumen in the outer sheath. A first drive mechanically engages and drives the outer sheath, and a second drive mechanically engages and drives the inner member through the slot and into the lumen of the outer sheath. Accordingly, the outer sheath and the inner member can be advanced separately or together.

Some embodiments of a method of this invention provide for advancing and retracting of inner and outer members of telescoping medical devices. One embodiment of a method provides for movement within a subject's body of an elongate, telescoping medical device having an outer sheath and an inner member. The method includes engaging and selectively advancing and retracting the outer sheath to selectively move the distal end of the outer sheath in the subject's body, and engaging and selectively advancing and retracting the inner member extending through an opening in the outer sheath intermediate the proximal and distal ends of the outer sheath. The inner member extends through the opening and a lumen in the outer sheath, so as to allow for selectively moving the distal end of the inner member in the subject's body.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a top view of one embodiment of an advancer system for coaxial medical devices, with the outer coaxial member shown in cross-section, according to the principles of this invention;

FIG. 2 is a top view of the advancer system of FIG. 1, with the outer coaxial member shown in cross-section, and with the outer member of the coaxial device advanced relative to the inner member;

FIG. 3 is a top view of a second embodiment of an advancer system for coaxial medical devices, with the outer coaxial member shown in cross-section;

FIG. 4 is a top view of the advancer system of FIG. 2, with the outer coaxial member shown in cross-section, and with the inner and outer members of the coaxial device advanced;

FIG. 5 is a top view of a third embodiment of an advancer system for coaxial medical devices, with the outer coaxial member shown in cross-section, according to the principles of this invention;

FIG. 6 is a top view of the advancer system of FIG. 5, with the outer coaxial member shown in cross-section, and with the inner and outer members of the coaxial device advanced;

FIG. 7 is a top view of the advancer system of FIG. 5, with the outer coaxial member shown in cross-section, and with the inner member of the coaxial device advanced relative to the outer member;

FIG. 8 is a top view of one embodiment of an advancer system, adapted for use with a telescoping device including an outer coaxial member having a seam adapted to receive an inner member, according to the principles of this invention;

FIG. 9 is a perspective view of an embodiment of a telescoping medical device according to the principles of this invention;

FIG. 10 is a side elevation view of an alternate construction of the device shown in FIG. 9;

FIG. 11 is a transverse cross-sectional view of the device of FIG. 10;

FIG. 12 is a side elevation view of another embodiment of the device shown in FIG. 10;

FIG. 13 is a side elevation view of another embodiment of a telescoping medical device;

FIG. 14 is a transverse cross sectional view of the device of FIG. 13;

FIG. 15 is a side elevation view of another embodiment of a telescoping medical device;

FIG. 16 is a side elevation view of another embodiment of a telescoping medical device;

FIG. 17 is a side elevation view of another embodiment of a telescoping medical device;

FIG. 18 is a side elevation view of another embodiment of a telescoping medical device having a intermittent seam;

FIG. 19 is a side elevation view of another embodiment of a telescoping medical device having a intermittent seam;

FIG. 20 is a side elevation view of another embodiment of a telescoping medical device having a intermittent seam;

FIG. 21 is a side elevation view of another embodiment of a telescoping medical device having a intermittent seam;

FIG. 22 is a top plan view of a system for operating an advancer mechanism for controlling the deployment of a medical device in accordance with the principles of the present invention.

Corresponding reference numerals indicate corresponding parts throughout the several views of the drawings.

### DETAILED DESCRIPTION

The following description is merely exemplary in nature and is not intended to limit the present disclosure, application, or uses. It should be understood that throughout the drawings, corresponding reference numerals indicate like or corresponding parts and features.

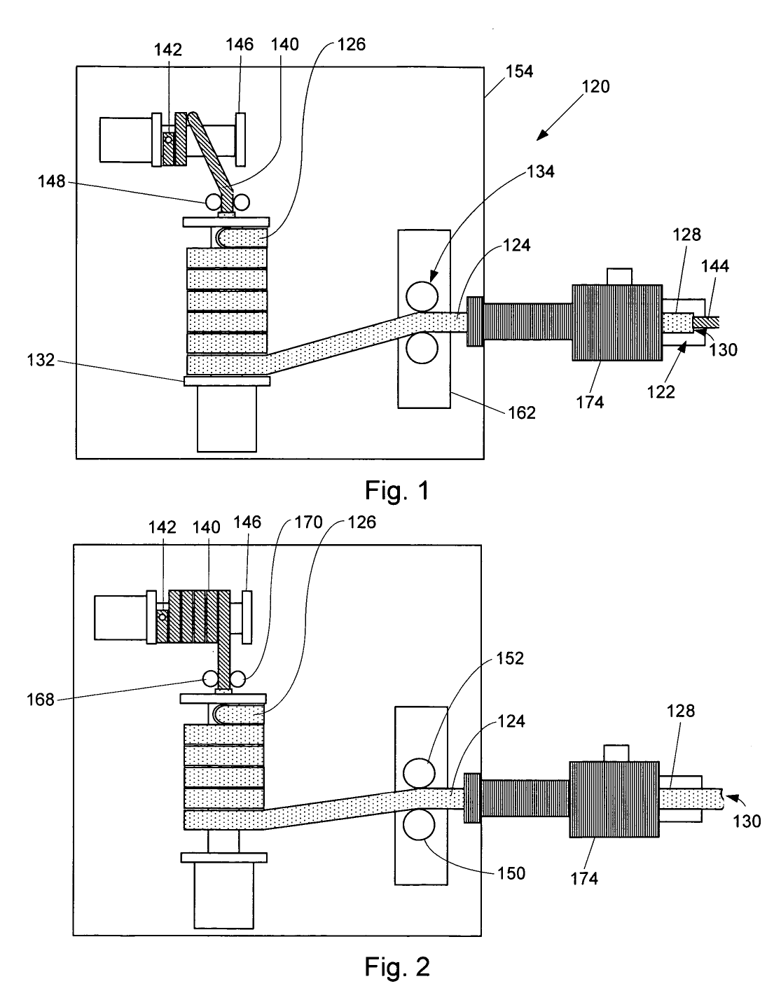

A first embodiment of an advancer mechanism in accordance with the principles of this invention is indicated generally as 120 in FIGS. 1 and 2. The advancer mechanism 120 is adapted for selectively advancing a flexible outer member 124 and a flexible inner member 140 of a telescoping medical device 122. The medical device 122 comprises a flexible outer sheath 124, having a proximal end 126, and distal end 128, and a lumen 130 therebetween. The advancer mechanism may further comprise an introducer 136 for insertion of the medical device 122 into a patient's body.

A flexible inner member 140, having a proximal end 142 and a distal end 144, telescopes axially in the lumen 130 of the sheath 124, and is coiled about a first winch spool 146. From the first winch spool 146, the inner member 140 is received within the outer member or sheath 124, having a generally hollow lumen 130 therein. The inner member 140 extends through a length of the sheath 124 that is coiled around a second winch spool 132. The inner member 140 may be deployed through the outer sheath 124 into a patient's body by controlling the first winch 146 and a first set of rollers 148. The first set of rollers includes a drive roller or wheel 150 and a driven wheel 152 that engage the inner member 140, such that rotation of the drive wheel 150 advances and retracts the inner member 140. Accordingly, the first winch 146 and the first set of rollers 148 may feed a length of the inner member 140 through the outer sheath 124 and into a patient's body as required to reach a desired area within the patient's body.

The second winch spool 132 may be rotated, along with a second set of rollers 134, to feed the outer sheath 124 over the deployed inner member 140 which has been deployed within the patient's body and is generally stationary. The second set of rollers includes a drive roller or wheel 168 and a driven wheel 170 that engage the outer sheath 124, such that rotation of the drive wheel 168 advances and retracts the outer sheath 124. As the outer sheath 124 is fed out over the inner member 140 deployed within the body as shown in FIG. 2, the length of outer sheath 124 coiled around the second winch spool 132 decreases from that in FIG. 1. Since the length of the inner member 140 originally disposed within the coiled sheath 124 does not deploy while the sheath 124 is being dispensed, the difference between the original length of the inner member 140 and the reduced length of the outer sheath 124 is taken up by the first winch spool 142 as shown in FIG. 2. Thus, a portion of the inner member 140 is coiled about the first winch 142 while the outer sheath 124 is being advanced relative to the inner member 140 and into the patient.

Relative movement between the outer sheath 124 and the inner member 140 may be accomplished by turning the winch or reel 132 and either holding the reel 146 still, turning the reel 132 in the opposite direction, or turning the reel 146 in the same direction but at a slower rate. More specifically, to advance the inner member 140 into the subject relative to the outer sheath, the reel 146 is turned in a direction to advance the inner member 140 into the proximal end of the outer sheath 124. The reel 132 may be held still, or it can be turned in a direction opposite from the direction of reel 146, or it can even be turned in the same direction as reel 146, but at a slower rate. To retract the inner member 140 from the subject relative to the outer sheath, the reel 146 is turned in a direction to retract the inner member 140 from the proximal end of the outer sheath 124. The reel 132 may be held still, it can be turned in a direction opposite from the direction of reel 146, or it can even be turned in the same direction as reel 146, but at a slower rate.

In this manner it is possible to provide multiple telescoping devices, and the inner member 140 could also be provided with a lumen for another device, which could likewise be provided on its own reel, and can be advanced and retracted relative to the other devices, by controlling the rotation of the reels. The device is still compact, because the reels are stacked upon each other, with the proximal end of each device centrally positioned in its respective reel. The first winch 146 and the first set of drive rollers 148, as well as the second winch 132 and the second set of drive rollers 134, may be simultaneously controlled to advance, as well as to retract, both the inner and outer members within a patient's body.

The inner member 140 may initially be advanced into a patient's body to reach a desired target area, and the outer sheath may subsequently be advanced over the inner member to the target area using the advancing apparatus. The outer sheath 124 may also include one or more functional components near the distal end 128, to provide treatment to a desired target area in the patient. Accordingly, the inner member and outer member may both be simultaneously advanced into a patient's body to reach a desired target area at the same time, and may also be simultaneously retracted from the patient's body at the same time.

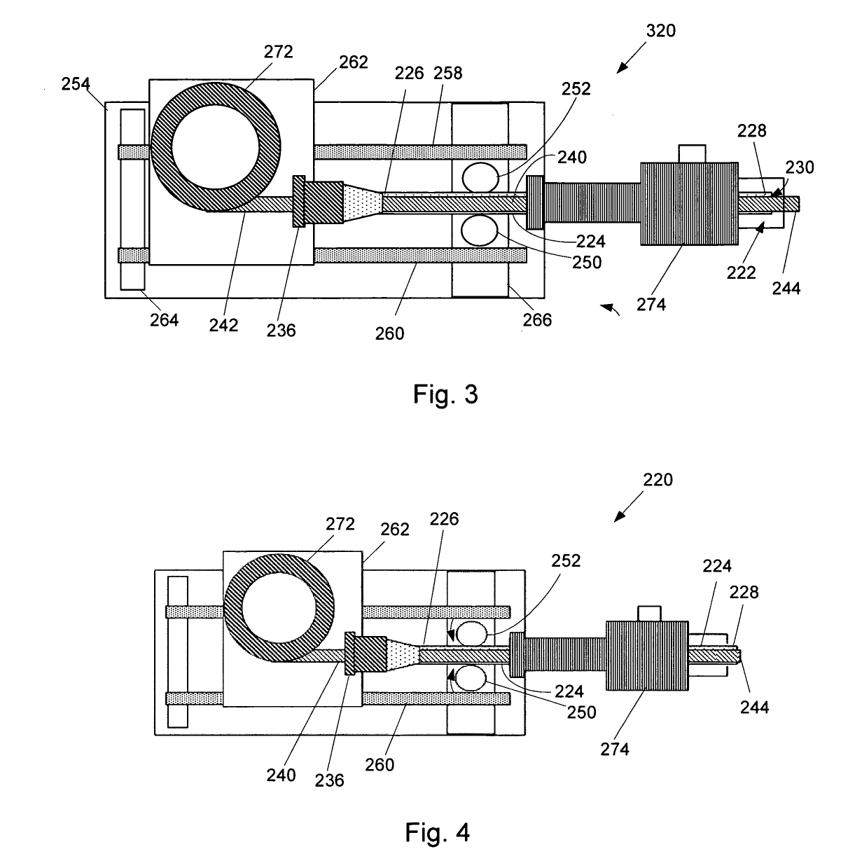

A second embodiment of an advancer mechanism is shown in FIGS. 3-4. The advancer mechanism 220 is adapted for selectively advancing the inner and outer members of a telescoping medical device 222. As shown in FIGS. 1-3, the medical device 222 comprises an outer sheath 224, having a proximal end 226, and distal end 228, and a lumen 230 there between. There is an adapter 236 on the proximal end of the outer sheath 224. The adapter 236 is provided for connection to a support block 262. An inner member 240, having a proximal end 242 and a distal end 244, telescopes axially in the lumen 230 of the sheath 224.

As shown in FIG. 3, advancer mechanism 220 comprises a base 254, having supports 264 and 266 mounted thereon. A pair of rails 258 and 260 are mounted on the supports 264 and 266. A support block 262 is movably mounted on the rails 258 and 260. A pair of rollers or wheels 250 and 252 are provided for advancing or retracting the outer member 224. The wheels 250 and 252 are sufficiently spaced apart to frictionally engage the exterior surface of the outer sheath 224 while accommodating the inner member 240, to advance and retract the outer sheath 224 and an adapter 236 engaged thereon. The support block 262 has a socket for receiving and engaging the adapter 236 that is engaged on the proximal end 226 of the outer sheath 224. The advancement or retraction of the outer member or sheath 224 by the wheels 250 and 252 causes the support block 262 to slide on the rails 258 and 260. The drive wheel 250 is connected to an appropriate drive system, such as an electric motor (not shown), with a flexible rotational drive element (not shown).

The support block 262 carries a reel 272 on which the inner member 240 is wound. The reel 272 is carried on a spindle that can be driven with a motor (not shown), or remote electric motor such as an indexing motor (not shown) which is connected to the drive wheel or wheels via a suitable power train (also not shown) which may include a flexible rotational drive shaft, clutch, and/or transmission. The unwinding of the reel 272 (and operation of a second pair of drive rollers where employed) causes the inner member 240 to advance, and the winding of the reel 272 causes the inner member 240 to retract.

The rotation of the reel 272 advances and retracts the inner member 240 relative to the outer sheath 224. The advancement or retraction of the outer member 224 by the wheels 250 and 252 causes the support block 262 to slide on the rails 258 and 260 to advance the outer sheath 224 and the inner member 240. The reel 272 can be locked to restrict movement of the inner member 240 as the wheels 250 and 252 move the inner sheath 240, to advance both the inner and outer members. Likewise the wheels 250 and 252 can lock to restrict movement of the outer sheath 224 as the reel 272 advances and retracts only the inner member 240. The system 220 thus allows the outer sheath 224 and the inner member 240 to be advanced and retracted separately, or in tandem.

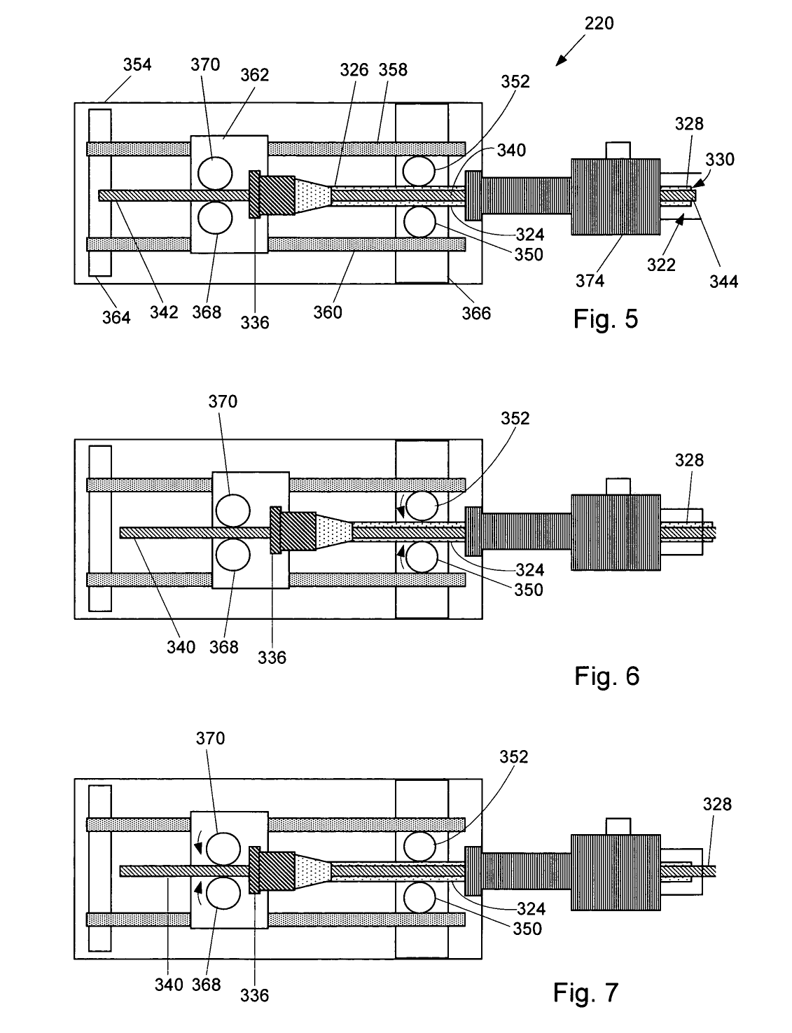

A third embodiment of an advancer mechanism is shown in FIGS. 5-7. The advancer mechanism 320 is adapted for selectively advancing the inner and outer members of a telescoping medical device 322. As shown in FIGS. 5-7, the medical device 322 comprises an outer sheath 324, having a proximal end 326, and distal end 328, and a lumen 330 there between. There is an adapter 336 on the proximal end of the outer sheath 324. The adapter 336 is provided for connection to a support block 362. An inner member 340, having a proximal end 338 and a distal end 340, telescopes axially in the lumen 330. The device 320 is similar in construction to the device 320, and corresponding parts are identified with corresponding reference numerals. Instead of a support block 362 that carries a reel 272, the advancer mechanism 320 has a support block 362 that carries a first set of rollers 368 and 370 on which the inner member 340 is advanced by.

As shown in FIG. 5, the advancer mechanism 320 comprises a base 354, having supports 364 and 366 mounted thereon. A pair of rails 358 and 360 are mounted on the supports 364 and 366. A support block 262 is slideably mounted on the rails 364 and 366. A driver comprising opposed drive wheel 350 and a driven wheel 352 engages the outer sheath 324, such that rotation of the drive wheel 350 advances and retracts the outer sheath 324. The drive wheel 350 is connected to an appropriate drive system, such as an electric motor (not shown), with a flexible rotational drive element (not shown). The wheels 350 and 352 are sufficiently spaced apart to frictionally engage the exterior surface of the outer sheath 324 while accommodating the inner member 340, to advance and retract the outer sheath 324 and an adapter 336 engaged thereon.

The support block 362 has a socket for receiving and engaging the adapter 336 engaged on the proximal end 328 of the outer sheath 324. The advancement or retraction of the outer member 324 by the wheels 350 and 352 causes the adapter 336 and support block 362 to move relative to rails 358 and 360. The support block 362 also includes a driver comprising opposed drive wheel 368 and a driven wheel 370 for engaging the inner member 340. Rotation of the drive wheel 368 advances and retracts the inner member 340 relative to the outer sheath 324. The drive wheel 368 is connected to an appropriate drive system, such as an electric motor (not shown), with a flexible rotational drive element (not shown). The wheels 368 and 370 are spaced sufficiently apart for frictionally engaging the exterior surface of the inner member 340 to advance and retract it through the outer sheath 324. The system 320 thus allows the outer sheath 324 and the inner member 340 to be advanced and retracted separately, or in tandem.

Operation

In operation, the outer sheath 324 is disposed between the wheels 350 and 352. The adapter 336 on the proximal end 326 of the outer sheath 324 is engaged in a socket in the support block 362. The inner member 340 is disposed between the wheels 368 and 370. Operating wheels 350 and 352 as shown in FIG. 6 causes the outer sheath 324, and the support block 362 to advance and retract. The wheels 368 and 370 can be driven in a direction opposite that of wheels 350 and 352, so that the outer sheath 324 can advance independent from the inner member 340. The wheels 368 and 370 also can be locked so that the outer sheath 324 can advance and retreat together with the inner member 340. Similarly, operating wheels 368 and 370 as shown in FIG. 7 causes the inner member 340 to advance and retract. Wheels 350 and 352 can be locked so that the inner member 340 can advance and retract independently of the outer sheath 324. The wheels 350 and 352 can be driven in conjunction with wheels 368 and 370 so that the outer sheath 324 can be advanced and retreated in tandem with the inner member 340.

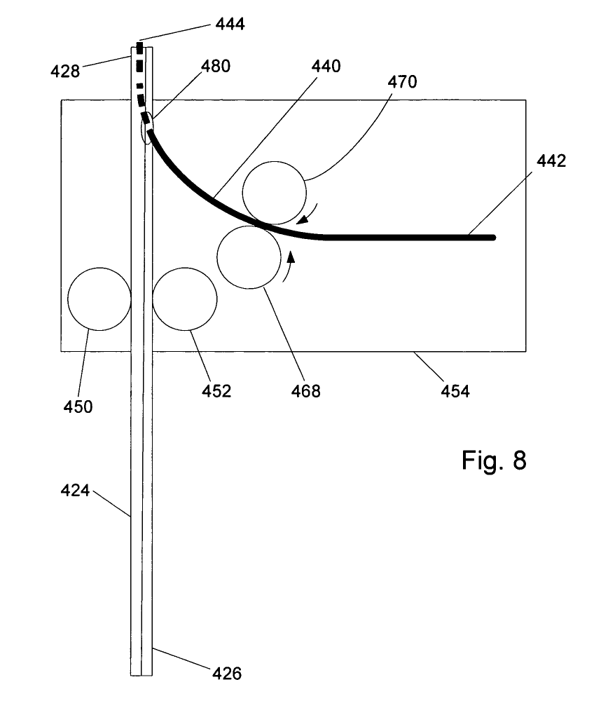

In fourth embodiment, an advancer mechanism and a system are provided for controlling coaxial medical devices. As shown in FIG. 8, a coaxial medical device 422 comprises an outer sheath 424 having a proximal end 426, a distal end 428, and a lumen 430 extending through at least a portion of the outer sheath 424. A driver comprising opposed drive wheel 450 and a driven wheel 452 engages the outer sheath 424, such that rotation of the drive wheel 450 advances and retracts the outer sheath 424. The drive wheel 450 is connected to an appropriate drive system, such as an electric motor (not shown), with a flexible rotational drive element (not shown). The wheels 450 and 452 are sufficiently spaced apart to frictionally engage the exterior surface of the outer sheath 424 while accommodating the inner member 440, to advance and retract the outer sheath 424.

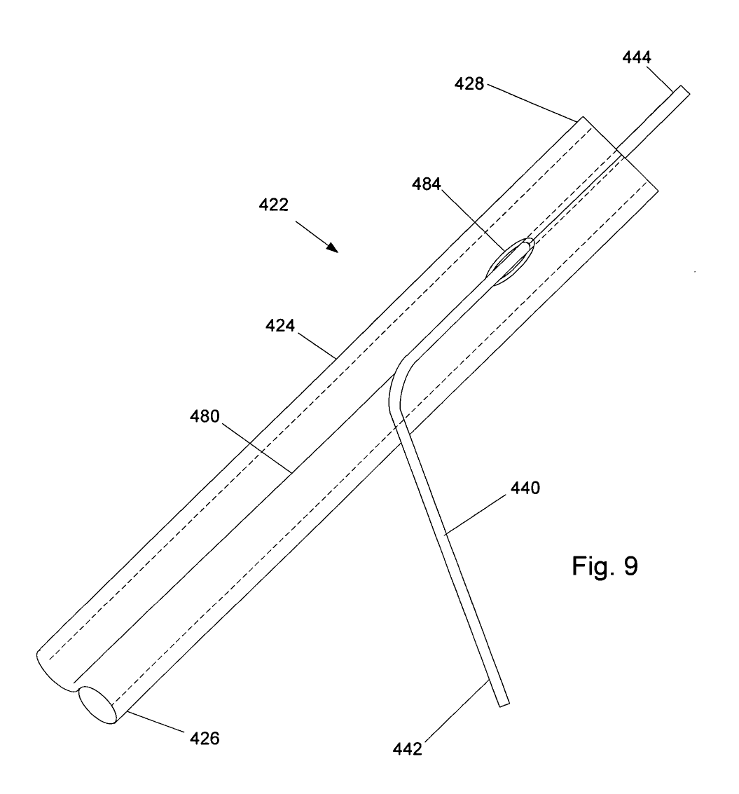

Referring to FIG. 9, the medical device 422 is shown in more detail. A slit 480 preferably extends along a distal portion of the outer sheath 424, including some or all of the portion containing the lumen 430, providing access to the lumen from outside the outer sheath. An inner member 440 having a proximal end 442 and a distal end 444, extends through the slit 480 at 482 and into the lumen 430 of the outer sheath 424. An eyelet 484 that can extend into the slit of the outer sheath 424, wedging the slit 480 open, to provide a gap through which the inner member 440 can pass, and to allow the slit to close smoothly. A driver comprising opposed drive wheel 468 and a driven wheel 470 engages the inner member 440, such that rotation of the drive wheel 468 advances and retracts the inner member 440. The drive wheel 468 is connected to an appropriate drive system, such as an electric motor (not shown), with a flexible rotational drive element (not shown). In this preferred embodiment, the wheels are driven by a remote electric motor (preferably a stepper motor) (not shown) which is connected to the drive wheel or wheels via a suitable power train (also not shown) which may include a flexible rotational drive shaft, clutch, and/or transmission. The wheels 468 and 470 are spaced sufficiently apart for frictionally engaging the exterior surface of the inner member 440 to advance and retract it through the outer sheath 424.

In the system for using an advancing apparatus, the drive mechanisms can be individually controlled by the physician with appropriate controls (not shown). Alternatively, the control of the drive mechanisms can be combined into a single control, which may be used direction by the physician, or integrated into a computer control that that is part of a larger navigation system that can orient and advance a generally coaxial medical device. The inner and outer members of the coaxial medical device accordingly can be respectively controlled to advance the inner member, for example, to a target area within the patient's body, or to advance the outer member over the inner member, or to advance both the outer member and inner member together, for enabling the physician to employ the coaxial medical device within the patient's body.

In a first embodiment of a medical device, the medical device 422 comprises an inner member 440 that may be received within an outer sheath 424 having a slit 480 as shown in FIGS. 9-12. The slit 480 preferably extends along a distal portion of the outer sheath 424, including some or all of the portion containing a lumen 430, and provides access to the lumen from outside the outer sheath. An eyelet 484 that can extend into the slit of the outer sheath 424, wedging the slit 480 open, to provide a gap through which an inner member 440 can pass, and to allow the slit to close smoothly.

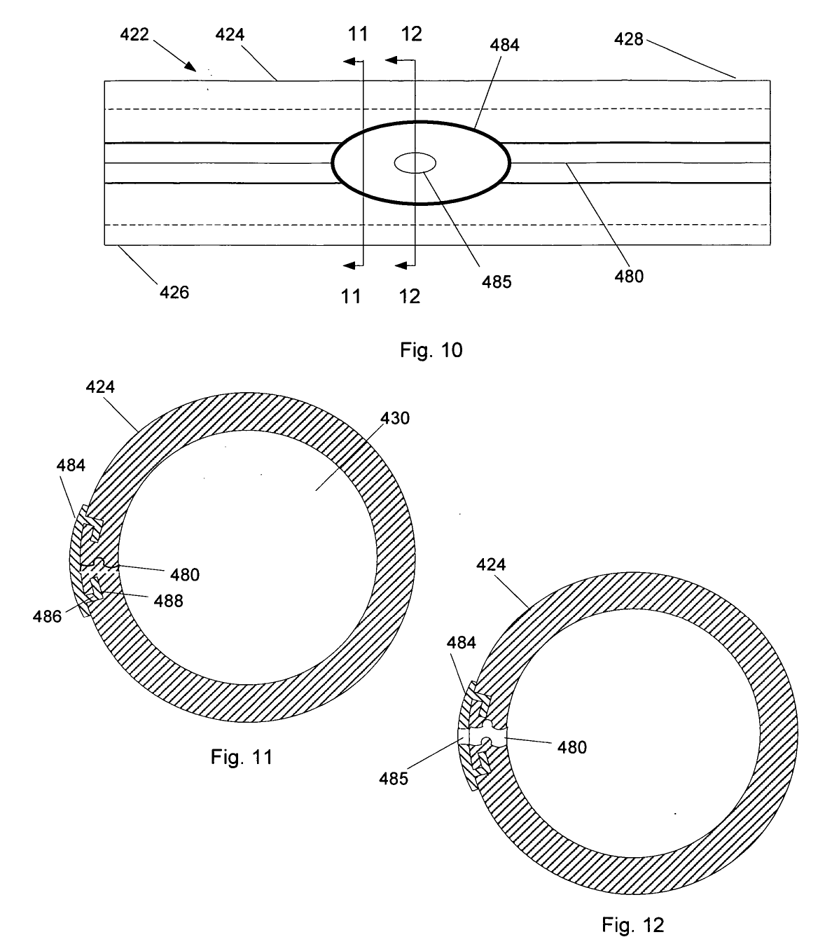

Referring to FIG. 10, the eyelet 484 is shown in more detail. The eyelet functions similar to that of a zipper, in separating the adjoining portions of the outer sheath 424 on either side of the slit 480. The eyelet 484 has a first cross-section shown in FIG. 11 that is adapted to holding the adjoining portions of the outer sheath 424 on either side of the slit 480 together. The cross-section includes track members 486 that are configured to be received within slots 488 in the outer sheath 424, which allow the eyelet to retain the adjoining portions of the outer sheath 424 together. The eyelet 484 has a second cross-section shown in FIG. 12 that is adapted to separate the adjoining portions of the outer sheath 424 on either side of the slit 480. The cross-section includes track members 486 that are configured to be received within slots 488 and 490 in the outer sheath 424, and are spaced apart to enable the eyelet to separate the adjoining portions of the outer sheath 424. The eyelet 484 further includes an opening 485 through the cross-section shown in FIG. 12, through which an inner member 440 may be deployed.

As shown in FIGS. 11 and 12, the adjoining margins 492 and 494 of the slit 480 in the outer sheath 424 have an interfitting configuration, for example with interfitting ribs of the type found on recloseable plastic bags, so that the slit can be opened and closed. The slit 480 can be opened so that an inner member of the medical device can extend through the slit 480 and into the lumen 430. The surrounding portions of the slit 480 (at least the portions of distal to the point of insertion of the inner member) are closed to retain the inner member in the lumen 430. There is also a “zipper” eyelet element 484 that slides in grooves or slots 488 and 490, which can engage and disengage the interfitting structures on the margins 492 and 494. Grooves 488 and 490 can be formed on the surface of the outer sheath 102d, and can be gripped to engage and disengage the interfitting ribs on the margins 492 and 494. The slit 480 can be opened so that the inner member 440 can extend through the slit 480 and into the lumen 430. The surrounding portions of the slit 480 (at least the portions of distal to the point of insertion of the inner member) are engaged to retain the inner member 440 in the lumen 430. The eyelet 484 can be formed with an opening 485 aligned with the slit 480, and the eyelet 484 opens the slit 480 in front of it, and closing it behind it, so that the slit remains closed, except in the vicinity of the eyelet 484, and the opening 485 allows the inner member 440 to pass through the wall of outer sheath 424 and into the lumen 430. The closing of the longitudinal slot accordingly comprises mechanically engaging mating elements on the opposed edge margins of the slot, such as with an engagement member that in sliding fashion engages mating elements on the opposed edge margins of the slot.

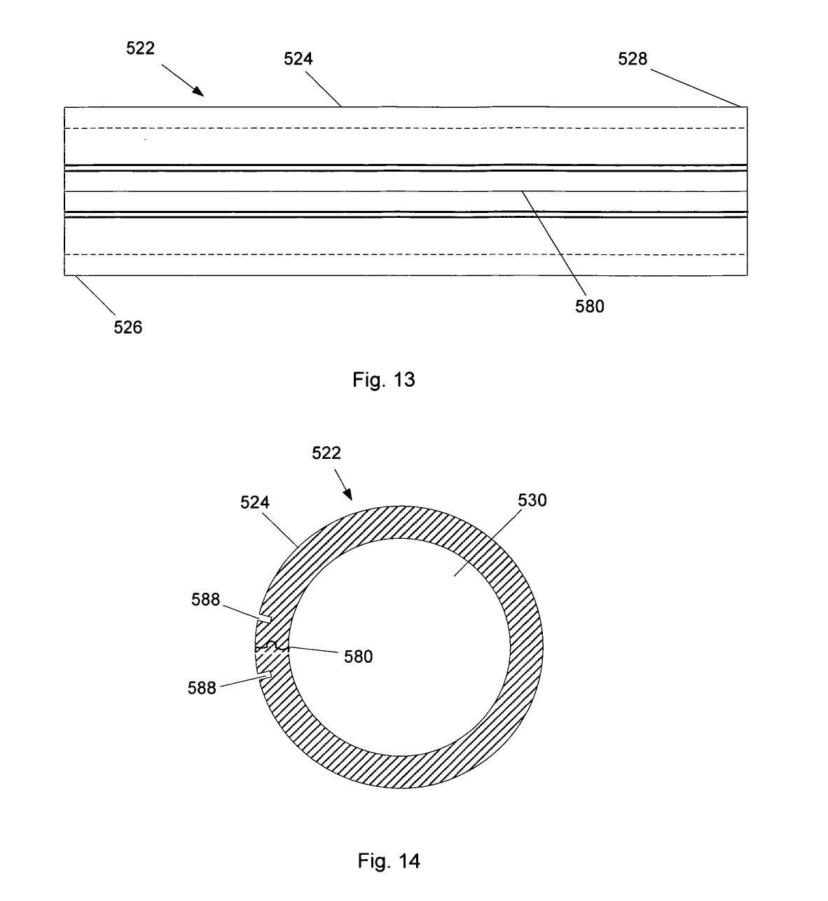

In a second embodiment of a medical device 522 shown in FIGS. 13 and 14, the device is similar in construction to device 422, and corresponding parts are identified with corresponding reference numerals. The medical device 522 includes an outer member 524 having a slit 580 therein, where an eyelet (not shown) that can extend into the slit of the outer sheath 524, wedging the slit 580 open to provide an opening through which an inner member can pass. However as shown in FIGS. 13 and 14, the outer sheath 524 includes a simple groove or slot 588 on both sides of a slit 480 in the outer sheath 524. The slots 588 are generally parallel to the slit 580 in the outer sheath 524, and are configured to receive track members of an eyelet. The spacing between such track members may vary to separate the adjoining portions of the outer sheath 524 on either side of the slit 580, as well as to join the portions and to allow the slit 580 to close smoothly. The outer member or sheath 624 may accordingly be used with an eyelet 584 to establish an opening in the outer member 624 through which an inner member may be deployed.

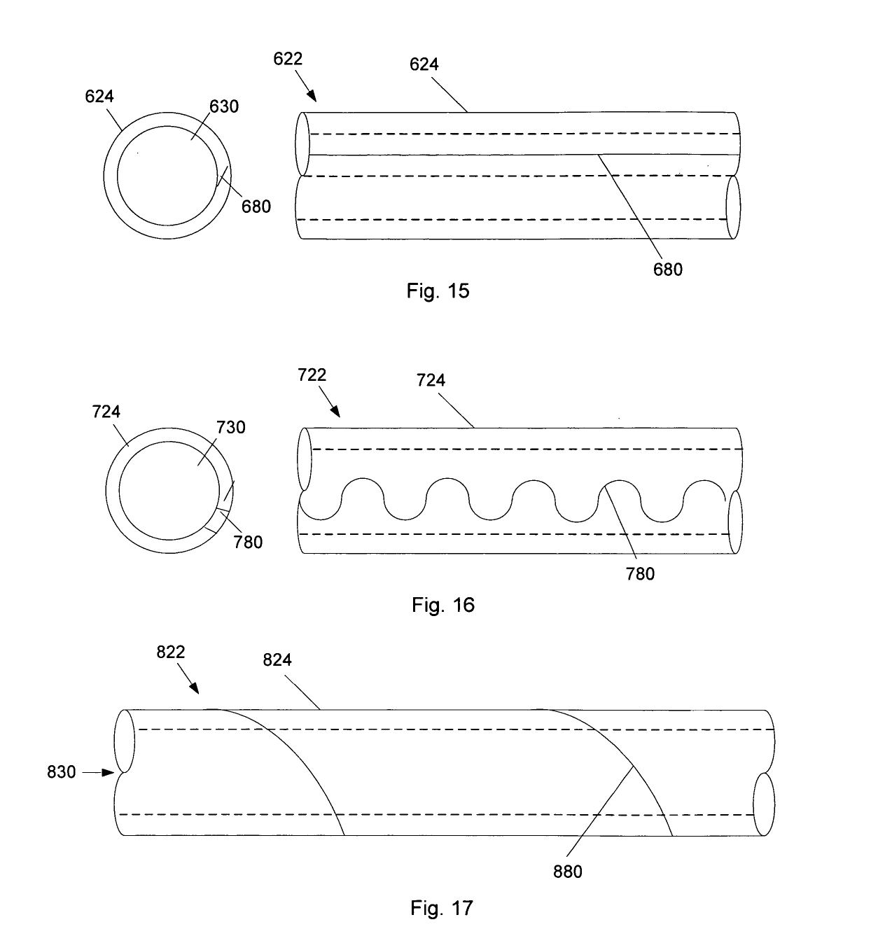

In a third embodiment of a medical device 622, the device 622 is similar in construction to device 422, and corresponding parts are identified with corresponding reference numerals. The medical device 622 includes an outer member 624 having a slit 680 therein, where an eyelet (not shown) that can extend into the slit of the outer sheath 624, wedging the slit 680 open to provide an opening through which an inner member can pass. However as shown in FIG. 15, the slit 684 in the outer sheath 624 has a lapped configuration in which the slit 684 extends through the wall of the outer sheath 624 at an angle with respect to the radial direction R, as shown in FIG. 15. This configuration helps to retain an inner member 640 in a lumen 630 of the outer sheath 624, particularly as the outer sheath 624 bends as it is navigated through a subject's body. However, this configuration still relatively easily permits the inner member 640 to pass into the lumen 630 of the outer sheath 624.

A fourth embodiment of a medical device 722 is shown in FIG. 16. Device 722 is similar in construction to device 422, and corresponding parts are identified with corresponding reference numerals. The medical device 722 includes an outer member 724 having a slit 780 therein, where an eyelet (not shown) that can extend into the slit of the outer sheath 724, wedging the slit 780 open to provide an opening through which an inner member can pass. However as shown in FIG. 16, the slit 780 in the outer sheath 724 has a wavy or crenulated configuration in which the slit 780 extends through the wall of the outer sheath 724 in generally sinusoidal pattern. This configuration helps to retain an inner member of the medical device within the lumen 730 of the outer sheath 724, particularly as the outer sheath bends as it is navigated through a subject's body. However, this configuration still relatively easily permits the inner member to pass into the lumen 730.

A fifth embodiment of a medical device 822 is shown in FIG. 17. Device 822 is similar in construction to device 422, and corresponding parts are identified with corresponding reference numerals. The medical device 822 includes an outer member 824 having a slit 880 therein, where an eyelet (not shown) that can extend into the slit of the outer sheath 824, wedging the slit 880 open to provide an opening through which an inner member can pass. However as shown in FIG. 17, the slit 880 in the outer sheath 824 has a spiral or helical configuration in which the slit 880 extends through the wall of the outer sheath 824 and winds around the circumference of the outer sheath. This configuration helps to retain an inner member of the medical device in the lumen 830 of the outer sheath 824, particularly as the outer sheath bends as it is navigated through a subject's body. However, to facilitate inserting and removing the inner member of the medical device into the lumen 830, the eyelet mechanism could revolve around the outer sheath 824, but that is relatively difficult. Alternatively, the outer sheath 824 could be rotated as it is advanced and retracted in a manner coordinated with the pitch of the spiral of the slot 880, so that the insertion point of the inner member through an eyelet into the lumen 830 stays in substantially the same place, so that the relative locations of the eyelet and advancement mechanisms can remain unchanged.

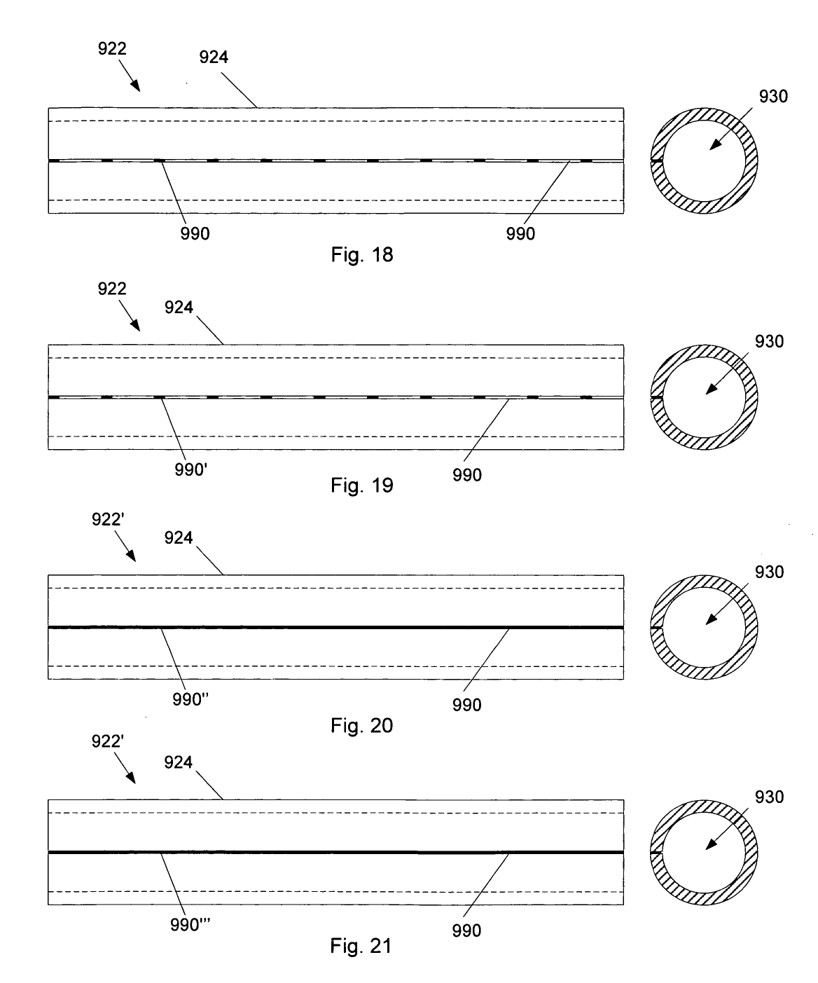

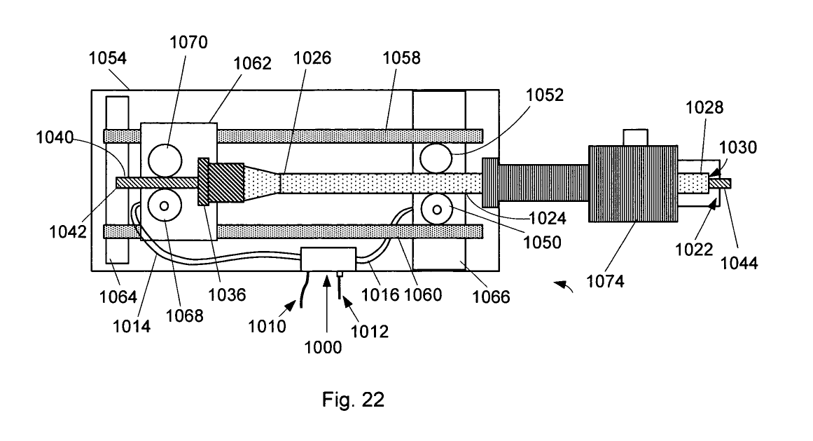

A fifth embodiment of a medical device 922 is shown in FIGS. 18-21. Device 922 is similar in construction to device 422, and corresponding parts are identified with corresponding reference numerals. The medical device 922 includes an outer member 924 having a slit 980 therein, where the adjoining portions of the outer sheath 924 on each side of the slit 980 may be intermittently bonded together at 990 as shown in FIG. 18. As the outer member 980 is advanced or retracted, an applicator may break intermittent bonds 990 to separate the adjoining portions of the outer sheath 924 to open the slit 980 to provide a space through which an inner member can pass. The applicator may also form intermittent welds 990 to re-seal the outer sheath 924, and may employ a heat welder or an ultrasonic welder that applies high frequency pressure to the outer member to intermittently bond the adjoining portions of the outer sheath 924. Similarly, the adjoining portions of the outer sheath 924 on each side of the slit 980 may be intermittently glued together at 990′ as shown in FIG. 19. As the outer member 980 is advanced or retracted, an applicator may break the intermittent glued portions 990′ to open the slit 980 to provide a space through which an inner member can pass, and may also intermittently apply adhesive or glue to re-seal the outer sheath 924. For example, the closing of the longitudinal slot may comprise ultravioletly actuating an adhesive on the edge margins of the slot. In this manner, an applicator may be employed to separate and intermittently bond the adjoining portions of the outer sheath 924 along the slit 980 to permit an inner member to be deployed within the lumen 930 of the outer sheath 924.

In an alternate construction of the medical device 922′, the medical device 922′ includes an outer member 924 having a slit 980 therein, where the adjoining portions of the outer sheath 924 on each side of the slit 980 may be continuously bonded together at 990″. An applicator may be employed to separate and bond the adjoining portions of the outer sheath 924 along the slit 980 to permit an inner member to be deployed within the lumen 930 of the outer sheath 924. The applicator may apply an adhesive, or may ultravioletly actuate an adhesive on the edge margins of the slot to form the continuous bond 990″ as in FIG. 20. Alternatively, the applicator may repeatedly apply high frequency ultrasonic pressure to the outer member to form an ultrasonic weld or bond 990′″ between the adjoining portions of the outer sheath 924 as in FIG. 21.

A method is also provided for moving in a subject's body an elongate, telescoping medical device that comprising at least an outer sheath and an inner member. The method comprises engaging and selectively advancing and retracting the outer sheath to selectively move the distal end of the outer sheath in the subject's body. The method also provides for engaging and selectively advancing and retracting the inner member, which extends through an opening in the outer sheath intermediate the proximal and distal ends of the outer sheath and into a lumen in the sheath. The inner member is advanced and retracted through the outer sheath to selectively move the distal end of the inner member in the subject's body. The opening in the outer member or sheath moves relative to the outer member as the outer member is advanced and retracted, while the opening remains relatively stationary relative to the subject's body. The method provides for forming an opening in the outer sheath for the inner member to pass through, by wedging open a generally longitudinally extending slot in the outer sheath. The outer sheath is engaged and selectively advanced and retracted at a point proximal to the opening in the sheath.

In a control system 1000 for controlling an advancing apparatus for a coaxial medical device, such as the various embodiments described herein, the drive mechanisms 1050, 1052 and 1068, 1070 can be individually controlled by the physician with appropriate controls (not shown). The control system 1000 is preferably connected to power via 1010 for actuating the drive mechanisms. The control system 1000 may receive information relating to the advancement or retraction of the medical device, through a data input 1012 that is in communication with one or more input devices. Such input devices may include but are not limited to computer keyboards, joysticks, or even voice activated devices. The control system receives information (such as digital information transmitted to the control system) requesting the advancement or retraction of either the inner member and the outer member, or both, and the extent of advancement or retraction. The control system may receive a request to advance only the inner member 1040 a given distance, and may accordingly control application of power via 1014 to effect rotation of a motor (not shown) to operate the drive mechanisms 1068 and 1070 to advance the inner member 1040 the given distance. Similarly, the control system may receive a request to advance only the outer sheath 1024 a given distance, and may accordingly control application of power via 1016 to effect rotation of a motor (not shown) or to operate the drive mechanisms 1050 and 1052 to advance the outer sheath 1024, and control rotation of a motor to operate the drive mechanisms 1068 and 1070 to retract the inner member 1040 while the outer sheath is being advanced.

As the physician orients or guides the distal end of the medical device within a patient's body towards a target area within the body, the physician may provide inputs to the control system for operating the drive mechanisms 1050, 1052 and 1068, 1070 to advance or retract the inner member 1040 and the outer sheath 1024 as required. Such input devices may include but are not limited to computer keyboards, joysticks, or even voice activated devices. The drive mechanisms 1050, 1052 and 1068 and 1070 may be mounted on a removable cover or support, such that various drive mechanisms may be modularly installed and employed. Such modular drive mechanisms allow for appropriately spaced drive wheels to accommodate changes in diameter of the medical device's inner or outer member. The drive mechanisms mounted on a cover or support may be fit onto the base, and may cooperate with a precise drive mechanism for controlling the displacement of the inner and outer members.

The control system may also be controlled by or cooperate with a navigation system for guiding the distal end of the medical device. Such a system may allow the physician to orient the distal end of the medical device through the application of a magnetic field external to the patient, for orienting the distal end of the medical device in a desired direction. The control system 1000 for the advancer 1020 may cooperate with or receive input signals from such navigation systems to advance the inner or outer members when the distal end has been oriented. The navigation system may also receive other additional input signals, such as an ECG signal of a patient's heart, which signal may be used to gate a switching device on and off for advancing and retracting the medical device a small distance, to accommodate the beating of the patient's heart for maintaining the medical device in contact with the heart surface. Such an advancer device may be operated to advance and retract the medical device synchronous with the heart to enhance contact of the distal end with the heart tissue.

Alternatively, the control of the drive mechanisms can be combined into a single control, which may be used direction by the physician, or integrated into a computer control that that is part of a larger navigation system that can orient and advance a generally coaxial medical device. The single control may comprise a keyboard, a joystick, or other peripheral device or any combination of input devices for enabling the physician to advance or retract the medical device. The inner and outer members of the coaxial medical device accordingly can be respectively controlled to advance the inner member, for example, to a target area within the patient's body, or to advance the outer member over the inner member, or to advance both the outer member and inner member together, for enabling the physician to employ the coaxial medical device within the patient's body.

What is claimed:

1. A method of moving an elongate, telescoping medical device comprising at least an outer sheath and an inner member in a subject's body, the method comprising engaging and selectively advancing and retracting the outer sheath to selectively move the distal end of the outer sheath in the subject's body, and engaging and selectively advancing and retracting the inner member extending through an opening in the outer sheath intermediate the proximal and distal ends of the outer sheath and through a lumen in the outer sheath to selectively move the distal end of the inner member in the subject's body.

2. The method according to claim 1 wherein the opening in the outer member moves relative to the inner member as the outer member advances and retracts.

3. The method according to claim 2 further comprising forming an opening for the inner member by wedging open a generally longitudinally extending slot in the outer sheath.

4. The method according to claim 3 wherein the outer sheath is engaged and selectively advanced and retracted at a point proximal to the opening in the sheath.

5. The method according to claim 3 further comprising closing the longitudinal slit distal to the opening.

6. The method according to claim 5 wherein the step of closing the longitudinal slot comprises adhering the margins of the slot together with adhesive.

7. The method according to claim 5 wherein the step of closing the longitudinal slot comprises ultrasonically welding the edge margins of the slot.

8. The method according to claim 5 wherein the step of closing the longitudinal slot comprises ultraviolet activation of adhesive on the edge margins of the slot.

9. The method according to claim 5 wherein the step of closing the longitudinal slot comprises mechanically engaging mating elements on the opposed edge margins of the slot.

10. The method according to claim 5 wherein the step of closing the longitudinal slot comprises sliding an engagement member to engage mating elements on the opposed edge margins of the slot.

11. The method according to claim 5 wherein the slot is surrounded by sloping sidewalls forming a generally V-shaped cross-section.

12. The method according to claim 5 wherein the slot has a spiral configuration.

13. The method according to claim 5 wherein the slot has a wavy configuration.

14. The method according to claim 5 wherein the slot extends at and angle with respect to the radial direction, forming a lapped configuration.

15. A telescoping medical device system comprising a outer sheath, having a proximal end and a distal end, a lumen extending through at least the distal end portion, and a slot extending along the distal end portion of the outer sheath, and an inner member extending through the slot and through the lumen in the outer sheath;
a first drive mechanically engaging and driving the outer sheath;
a second drive mechanically engaging and driving the inner member through the slot and into the lumen of the outer sheath; so that the outer sheath and the inner member can be advanced separately or together.

16. The system of claim 15, where at least one of the inner or outer medical device members that is driven passes across a roller in a generally helically wound fashion.

17. The system according to claim 15 further comprising a wedge extending though the slot and forming an opening in the slot to permit the passage of the inner member.

18. The system according to claim 15 further comprising an applicator for applying adhesive to the margins of the slot distal to the opening to hold the margins together.

19. The system according to claim 15 further comprising ultrasonic welding for welding the margins of the slot distal to the opening to hold the margins together.

20. The system according to claim 15 further comprising an ultraviolet light source for curing an adhesive on the margins of the slot distal to the opening to hold the margins together.

21. The system according to claim 15 further comprising means for forming an opening for the inner member by wedging open a generally longitudinally extending slot in the outer sheath.

22. The system according to claim 15 wherein the outer sheath is engaged and selectively advanced and retracted at a point proximal to the opening in the sheath.

23. The system according to claim 15 further comprising means for closing the longitudinal slit distal to the opening.

24. The system according to claim 15 wherein the means for closing the longitudinal slit comprises means for adhering the margins of the slot together with adhesive.

25. The system according to claim 15 wherein the means for closing the longitudinal slit comprises means for ultrasonically welding the edge margins of the slot.

26. The system according to claim 15 wherein the means for closing the longitudinal slit comprises means for ultraviolet activation of adhesive on the edge margins of the slot.

27. The system according to claim 15 wherein the means for closing the longitudinal slit comprises means for mechanically engaging mating elements on the opposed edge margins of the slot.

28. The system according to claim 15 wherein the means of closing the longitudinal slot comprises sliding an engagement member to engage mating elements on the opposed edge margins of the slot.

29. The system according to claim 15 wherein the slot is surrounded by sloping sidewalls forming a generally V-shaped cross-section.

30. The system according to claim 15 wherein the slot has a spiral configuration.

31. The system according to claim 15 wherein the slot has a wavy configuration.

32. The system according to claim 15 wherein the slot extends at and angle with respect to the radial direction, forming a lapped configuration.
